(19) United States
(12) Patent Application Publication
(10) Pub. No.: US 2008/0033192 A1
(43) Pub. Date: Feb. 7, 2008

(54) Process for the Production of Esters from Vegetal Oils or Animal Fats

(75) Inventors: Dante Siano, Cologno Monzese (IT); Martino Di Serio, Cava Dei Tirreni (IT); Riccardo Tesser, Caserta (IT); Marinella Dimiccoli, Gragnano (IT); Francesco Cammarota, Cava Dei Tirreni (IT); Elio Santacesaria, Milano (IT); Luigi Siano, Cologno Monzese (IT); Mario Nastasi, Marconia (IT)

Correspondence Address: Modiano & Associati, Via Meravigli 16, Milano, 20123, IT

(73) Assignee: ASER S.R.L., Cernusco Sul Naviglio (IT)

(21) Appl. No.: 11/630,347

(22) PCT Filed: Jun. 29, 2005
(86) PCT No.: PCT/IB05/01865

§ 371 Date: Apr. 25, 2007

(30) Foreign Application Priority Data
Jun. 30, 2004 (IT) MI2004A001323

(51) Int. Cl. C07C 51/41

(52) U.S. Cl. 554/174

(57) ABSTRACT

A process for producing esters from vegetal oils and animal fats, comprising the step of transesterification of vegetal oils or animal fats by reaction with an alcohol with low molecular weight in the presence of a catalyst comprising a salt of a carboxylic acid with a metal, wherein the salt of a carboxylic acid with a metal is a salt of a carboxylic acid with a metal selected from the group consisting of metals having a stability constant of the complex with di-benzoyl-methane logβ_{DBM} in the range between 8.54 and 10.35, or is a salt with a metal of a carboxylic acid selected from the group consisting of fatty acids.

### TECHNICAL FIELD

The present invention relates to a process for the production of esters from vegetal oils or animal fats even in the presence of high concentrations of free fatty acids.

### BACKGROUND ART

Biodiesel, used as fuel in Diesel engines, is constituted by a mixture of esters of fatty acids, which can be obtained by a transesterification reaction of vegetal oils and animal fats with alcohols, particularly methanol or ethanol, and subsequent separation from glycerol.

The transesterification reaction for the production of biodiesel is generally performed by using as catalysts bases of alkaline metals, such as for example NaOH, KOH, NaOCH₃, KOCH₃ [1,2].

However, these catalysts cannot be used in the presence of humidity or if the acidity of the substrate to be subjected to transesterification is high due to a high content of free fatty acids. This often occurs if the oils and fats originate from production waste. In this case, the esters are generally produced by subjecting the oil or fat having a high content of free fatty acids, first to an esterification process, by using an acid catalyst, and then to the transesterification process by using the basic catalysts cited above [1,3,4].

An alternative is to use acid catalysts both for the esterification reaction and for the transesterification reaction. Y. Zhang et al., for example, have proposed a process in which by using an acid oil (1.5-3.5%) with methanol/oil ratio of 1.7:1 by weight and a catalyst (H₂SO₄)/oil ratio of 0.14:1 by weight at 80°, in 240 minutes an oil conversion equal to 97% is obtained [5]. A severe drawback of this process is the large amount of catalyst used in the reaction, which during the neutralization step produces high quantities of CaSO₄ (0.2 kg of salt per kilogram of oil used) [5].

Foglia et al. [6] have demonstrated that it is possible to perform transesterification of an acid oil by using the lipase enzyme as a catalyst. The reaction was found to be of limited industrial interest, since it requires 4-16 hours to achieve 95% conversion.

Basu and Norris, in order to obviate these drawbacks, have proposed a process for producing esters from oils with high free acidity in a single stage [7]. In the Basu and Norris patent [7] it is shown that a mixture of calcium acetate and barium acetate with a weight ratio of 3:1 provides high conversions to esters of oils and fats with an acidity of less than 10% by weight, by working for three hours at 200-250° C. and a catalyst/oil ratio by weight of approximately 0.005.

The process proposed by Basu and Norris [7], however, has the drawback that due to the high temperatures required (200-250° C.) in order to have high conversions of the free acid and of the oil to methyl ester, the operating pressures of the system are rather high (40-95 bars).

### DISCLOSURE OF THE INVENTION

The aim of the present invention is to provide a process for producing with a high yield esters from raw materials constituted by vegetal oils and animal fats by using, as raw material, both oils and fats that are not acid (free acidity <0.5% by weight (determined as weight of the oleic acid/weight of the oil) and oils and fats having a high acidity (free acidity >0.5% by weight).

An object of the present invention is to provide a process for producing esters from vegetal oils or animal fats with high transesterification conversions even in the presence of a substantial concentration of free acid, such as >1% by weight, at temperatures below 200° C.

Another object of the present invention is to provide a process for preparing esters from vegetal oils or animal fats at temperatures comprised between 200 and 250° C. by using low catalyst/oil weight ratios, for example <0.0005.

Further objects of the present invention will become better apparent from the detailed description of the invention.

This aim and other objects which will become better apparent from the the description that follows are achieved by a process according to the present invention for producing esters from vegetal oils and animal fats, which comprises the step of transesterification of vegetal oils or animal fats by reaction with an alcohol with low molecular weight in the presence of a catalyst comprising a salt of a carboxylic acid with a metal, the salt of a carboxylic acid with a metal being a salt of a carboxylic acid with a metal selected from the group consisting of metals having a stability constant of the complex with di-benzoyl-methane lOgPDBM in the range between 8.54 and 10.35, or being a salt with a metal of a carboxylic acid selected among the group consisting of fatty acids.

Preferably, the metal of the catalyst used in the present invention is selected from the group consisting of Mg, Cd, Mn, Pb, Zn, Co. More preferably, the metal is selected from the group consisting of Cd, Mn, Pb and Zn. The most preferred metal of the catalyst of the present invention is Pb.

In the embodiments of the present invention in which the catalyst is a salt of a carboxylic acid with a metal selected from the group consisting of metals with a stability constant of the complex with di-benzoyl-methane logβ_{DBM} in the range between 8.54 and 10.35, the carboxylic acid can be a non-fatty acid, such as acetic acid, or a fatty acid, preferably a C8-C22 fatty acid, and more preferably stearic acid.

In the embodiment of the present invention in which the catalyst comprises a salt with a metal of a fatty acid, for example stearic acid, the metal can be a divalent metal, for example Ca or Ba. Preferably, the metal is selected from the group consisting of Mg, Cd, Mn, Pb, Zn and Co. More preferably, the metal is selected from the group consisting of Cd, Mn, Pb and Zn. The most preferred metal of the catalyst of the present invention is Pb.

In a more preferred embodiment, the catalyst comprises lead stearate.

The alcohol used in the process of the present invention is preferably selected between ethanol and methanol.

The reaction of the process of the present invention occurs preferably at a temperature comprised between 100 and 260° C.

The process of the present invention has the advantage of allowing to have high transesterification conversions even in the presence of a substantial concentration of free acid at temperatures below 200° C. Moreover, at temperatures comprised between 200 and 250° C. it is possible to use catalyst/oil weight ratios <0.0005.

The raw material can be constituted by non-acid fats and oils (free acidity <0.5% by weight) and by oils and fats with high acidity (free acidity >0.5% by weight).

### BRIEF DESCRIPTIONS OF THE DRAWINGS

Further advantages will become evident from the detailed description of the invention. The invention is also described with reference to the following figures:

FIG. 1 Profile of the temperature and conversion of the oil to methyl ester as a function of the reaction time for the test of Example 7.

FIG. 2 Profile of the temperature and conversion of the oil to methyl ester as a function of the reaction time for the test of Example 8.

### WAYS OF CARRYING OUT THE INVENTION

It has been found that the activity of transesterification of vegetal oils and animal fats reaches a maximum value when the metallic cation of the catalyst has an acidity comprised in a very specific range. By quantifying the acidity of these metallic ions with the logarithm of their stability constant (logβ_{DBM}) with di-benzoyl-methane (DBM) [8,9], it has been found that the optimum range of the stability constant logβ_{DBM} is comprised between 8.54 (Mg⁺⁺) and 10.35 (Co⁺⁺).

Moreover, it has been found that the activity of the cations comprised in this range of the stability constant of the complex with DPM is significant also at temperatures below 150° C. In particular, it has been found that cations with a value of logβ_{DBM} comprised between 8.67 (Cd⁺⁺) and 10.23 (Zn⁺⁺) exhibit a higher activity than the other cations. The cation that has shown the highest activity is Pb⁺⁺(logβ_{DBM}=9.75).

The values of logβ_{DBM} have been determined by Van Uitert et al. [8].

Moreover, it has been found that in general, as the number of carbon atoms of the carboxylate anion increases, the transesterification activity of the corresponding catalyst increases: for example, conversions obtained with stearates are always higher than those obtained with acetates when using an equivalent molar quantity of catalyst.

The process claimed in the present invention can also be used for a raw material that has high concentrations of free acidity, for example more than 1% by weight of free acids.

The examples that follow are given as an illustration of the invention and must not be considered as limiting its scope.

All the reagents used were supplied by Fluka, except for soybean oil, supplied by Casa Olearia Italiana S.p.A. (Monopoli, BA).

### EXAMPLES

### Example 1

### Stearate Synthesis

Stearate synthesis is performed by reacting the corresponding acetates with stearic acid. Stoichiometric quantities of acetate and stearic acid are loaded into a round-bottomed flask; the system is kept at 180° C. for 3 hours and the resulting acetic acid is distilled. The conversion of acetate to stearate is calculated from the quantity of acetic acid obtained. Table 1 lists the conversions obtained for the various stearates used as catalysts in the examples that follow.

TABLE 1
Stearate synthesis

| Test | | |
|---|---|---|
| no. | Catalyst | Conversion (%) |
| 1.1 | Ba(OOC(CH₂)₁₆CH₃)₂ | 73 |
| 2.1 | Ca(OOC(CH₂)₁₆CH₃)₂ | 74 |
| 3.1 | Mg(OOC(CH₂)₁₆CH₃)₂ | 72 |
| 4.1 | Cd(OOC(CH₂)₁₆CH₃)₂ | 89 |
| 5.1 | Mn(OOC(CH₂)₁₆CH₃)₂ | 62 |
| 6.1 | Pb(OOC(CH₂)₁₆CH₃)₂ | 88 |
| 7.1 | Zn(OOC(CH₂)₁₆CH₃)₂ | 64 |
| 8.1 | Co(OOC(CH₂)₁₆CH₃)₂ | 81 |
| 9.1 | Ni(OOC(CH₂)₁₆CH₃)₂ | 67 |

### Example 2

### Tests for Transesterification of Oil with Low Content of Free Acidity with Acetates at 200° C. (Activity Comparison)

Reaction tests were conducted by loading into small steel reactors 2 g of soybean oil with an acidity of 0.2% by weight, 0.9 g methanol, and the catalyst. The reactors were placed in a ventilated oven and subjected to the following temperature program: 2 minutes at 30° C., heating at 20° C./min up to 200° C.; the reactors were held at this temperature for 55 minutes. The reactors were then cooled rapidly to ambient temperature.

The resulting conversions were determined by using H-NMR [10]. Table 2 lists the results obtained for the various tests.

TABLE 2
Oil: soybean oil; acidity: 0.2% by weight; temperature: 200° C.

| Test | | mol | logβ_{DBM} | Conversion |
|---|---|---|---|---|
| no. | Catalyst | M⁺⁺ ×10⁵ | [8, 9] | (%) |
| 1.2 | Ba(OOCCH₃)₂ | 5.71 | 6.10 | 11 |
| 2.2 | Ca(OOCCH₃)₂ | 5.77 | 7.17 | 31 |
| 3.2 | Mg(OOCCH₃)₂•4H₂O | 5.73 | 8.54 | 39 |
| 4.2 | Cd(OOCCH₃)₂ | 5.78 | 8.67 | 85 |
| 5.2 | Mn(OOCCH₃)₂ | 5.75 | 9.32 | 67 |
| 6.2 | Pb(OOCCH₃)₂ | 5.80 | 9.75 | 81 |
| 7.2 | Zn(OOCCH₃)₂•2H₂O | 5.83 | 10.23 | 67 |
| 8.2 | Co(OOCCH₃)₂•4H₂O | 5.77 | 10.35 | 20 |
| 9.2 | Ni(OOCCH₃)₂•4H₂O | 5.74 | 10.83 | 7 |
| 10.2 | Ca(OOCCH₃)₂ | 5.87 | — | 38 |
| | Ba(OOCCH₃)₂ | | | |
| | (Ca/Ba = 3/1 w/w) | | | |

As shown by the results given in the table, the best catalysts are acetates of cations characterized by a logβ_{DBM} comprised between 8.67 (Cd⁺⁺) and 10.23 (Zn⁺⁺). The activities of these catalysts are considerably higher than the activities of the calcium and barium acetates and of their mixture claimed by Basu and Norris [7].

### Example 3

### Tests for Transesterification of Oil with Low Content of Free Acidity with Stearates at 200° C. (Activity Comparison)

Reaction tests were conducted by loading into small steel reactors 2 g of soybean oil with an acidity of 0.2% by weight, 0.9 g of methanol, and the catalyst. The reactors were placed in a ventilated oven and subjected to the following temperature program: 2 minutes at 30° C., heating at 20° C./min up to 200° C.; the reactors were kept at this temperature for 55 minutes. The reactors were then cooled rapidly to ambient temperature.

The resulting conversions were determined by using H-NNR [10]. Table 3 lists the results obtained for the various tests.

TABLE 3
Oil: soybean oil; acidity: 0.2% by weight; temperature: 200° C.

| Test | | mol | logβ_{DBM} | Conversion |
|---|---|---|---|---|
| no. | Catalyst | M⁺⁺ ×10⁵ | [8, 9] | (%) |
| 1.3 | Ba(OOC(CH₂)₁₆CH₃)₂ | 5.74 | 6.10 | 57 |
| 2.3 | Ca(OOC(CH₂)₁₆CH₃)₂ | 5.77 | 7.17 | 67 |
| 3.3 | Mg(OOC(CH₂)₁₆CH₃)₂ | 5.75 | 8.54 | 57 |
| 4.3 | Cd(OOC(CH₂)₁₆CH₃)₂ | 5.73 | 8.67 | 88 |
| 5.3 | Mn(OOC(CH₂)₁₆CH₃)₂ | 5.76 | 9.32 | 81 |
| 6.3 | Pb(OOC(CH₂)₁₆CH₃)₂ | 5.72 | 9.75 | 92 |
| 7.3 | Zn(OOC(CH₂)₁₆CH₃)₂ | 5.72 | 10.23 | 72 |
| 8.3 | Co(OOC(CH₂)₁₆CH₃)₂ | 5.77 | 10.35 | 44 |
| 9.3 | Ni(OOC(CH₂)₁₆CH₃)₂ | 5.71 | 10.83 | 7 |

The results of the tests confirm that the activity of transesterification of oils and fats also for stearates has a maximum for cations characterized by a logβ_{DBM} comprised between 8.67 (Cd⁺⁺) and 10.23 (Zn⁺⁺). Moreover, by comparing the data of Table 2 with the values of Table 1, it can be said that stearates are in general more active than acetates.

### Example 4

### Tests for Transesterification of Oil with Low Content of Free Acidity with Acetates at 150° C. (Activity Comparison)

Reaction tests were conducted by loading into small steel reactors 2 g of soybean oil with an acidity of 0.2% by weight, 0.9 g of methanol, and the catalyst. The reactors were placed in a ventilated oven and subjected to the following temperature program: 2 minutes at 30° C., heating at 20° C./min up to 150° C.; the reactors were kept at this temperature for 55 minutes. The reactors were then cooled rapidly to ambient temperature.

The resulting conversions were determined by using H-NMR [10]. Table 4 lists the results obtained for the various tests.

TABLE 4
Oil: soybean oil; acidity: 0.2% by weight; temperature: 150° C.

| Test | | mol | logβ_{DBM} | Conversion |
|---|---|---|---|---|
| no. | Catalyst | M⁺⁺ ×10⁵ | [8, 9] | (%) |
| 1.4 | Ba(OOCCH₃)₂ | 5.76 | 6.10 | 1 |
| 2.4 | Ca(OOCCH₃)₂ | 5.83 | 7.17 | 2 |
| 3.4 | Mg(OOCCH₃)₂•4H₂O | 5.96 | 8.54 | 12 |
| 4.4 | Cd(OOCCH₃)₂ | 5.74 | 8.67 | 39 |
| 5.4 | Mn(OOCCH₃)₂ | 5.83 | 9.32 | 18 |
| 6.4 | Pb(OOCCH₃)₂ | 5.75 | 9.75 | 44 |
| 7.4 | Zn(OOCCH₃)₂•2H₂O | 5.79 | 10.23 | 30 |
| 8.4 | Co(OOCCH₃)₂•4H₂O | 5.78 | 10.35 | 6 |
| 9.4 | Ca(OOCCH₃)₂ | 5.93 | — | 2 |
| | Ba(OOCCH₃)₂ | | | |
| | (Ca/Ba = 3/1 w/w) | | | |

The results of the tests confirm that at 150° C. also, the activity of transesterification of oils and fats has a maximum for cations characterized by a logβ_{DBM} comprised between 8.67 (Cd⁺⁺) and 10.23 (Zn⁺⁺).

Moreover, the activities of these catalysts are considerably higher than the activities of calcium and barium acetates and of their mixture claimed by Basu and Norris [7], which at 150° C. have a distinctly negligible activity.

### Example 5

### Tests for Transesterification of Oil with Low Content of Free Acidity with Stearates at 150° C. (Activity Comparison)

Reaction tests were conducted by loading into small steel reactors 2 g of soybean oil with an acidity of 0.2% by weight, 0.9 g of methanol, and the catalyst. The reactors were placed in a ventilated oven and subjected to the following temperature program: 2 minutes at 30° C., heating at 20° C./min up to 150° C.; the reactors were kept at this temperature for 55 minutes. The reactors were then cooled rapidly to ambient temperature.

The resulting conversions were determined by using H-NMR [10]. Table 5 lists the results obtained for the various tests.

TABLE 5
Oil: soybean oil; acidity: 0.2% by weight; temperature: 150° C.

| Test | | mol | logβ_{DBM} | Conversion |
|---|---|---|---|---|
| no. | Catalyst | M⁺⁺ ×10⁵ | [8, 9] | (%) |
| 1.5 | Ba(OOC(CH₂)₁₆CH₃)₂ | 5.74 | 6.10 | 16 |
| 2.5 | Ca(OOC(CH₂)₁₆CH₃)₂ | 5.77 | 7.17 | 5 |
| 3.5 | Mg(OOC(CH₂)₁₆CH₃)₂ | 5.78 | 8.54 | 22 |
| 4.5 | Cd(OOC(CH₂)₁₆CH₃)₂ | 5.74 | 8.67 | 62 |
| 6.5 | Mn(OOC(CH₂)₁₆CH₃)₂ | 5.72 | 9.32 | 31 |
| 7.5 | Pb(OOC(CH₂)₁₆CH₃)₂ | 5.74 | 9.75 | 72 |
| 8.5 | Zn(OOC(CH₂)₁₆CH₃)₂ | 5.71 | 10.23 | 52 |
| 9.5 | Co(OOC(CH₂)₁₆CH₃)₂ | 5.72 | 10.35 | 11 |
| 9.5 | Ni(OOC(CH₂)₁₆CH₃)₂ | 5.72 | 10.83 | 11 |

The results of the tests confirm that the activity of transesterification of oils and fats, also for stearates at 150° C., has a maximum for cations characterized by a logβ_{DBM} comprised between 8.67 (Cd⁺⁺) and 10.23 (Zn⁺⁺). Moreover, by comparing the data of Table 3 and the values of Table 4 it can be said that stearates are in general more active than acetates also at 150° C.

### Example 6

### Tests for Transesterification of Oil with Low Content of Free Acidity with Stearates at 130° C. (Activity Comparison)

Reaction tests were conducted by loading into small steel reactors 2 g of soybean oil with an acidity of 0.2% by weight, 0.9 g of methanol, and the catalyst. The reactors were placed in a ventilated oven and subjected to the following temperature program: 2 minutes at 30° C., heating at 20° C./min up to 130° C.; the reactors were kept at this temperature for 55 minutes. The reactors were then cooled rapidly to ambient temperature.

The resulting conversions were determined by using H-NMR [10]. Table 6 lists the results obtained for the various tests.

TABLE 6
Oil: soybean oil; acidity: 0.2% by weight; temperature: 130° C.

| Test | | mol | logβ_{DBM} | Conversion |
|---|---|---|---|---|
| no. | Catalyst | M⁺⁺ ×10⁵ | [8, 9] | (%) |
| 1.6 | Mg(OOC(CH₂)₁₆CH₃)₂ | 4.60 | 8.54 | 4 |
| 2.6 | Cd(OOC(CH₂)₁₆CH₃)₂ | 5.39 | 8.67 | 33 |
| 3.6 | Mn(OOC(CH₂)₁₆CH₃)₂ | 4.71 | 9.32 | 16 |
| 4.6 | Pb(OOC(CH₂)₁₆CH₃)₂ | 4.67 | 9.75 | 48 |
| 6.6 | Zn(OOC(CH₂)₁₆CH₃)₂ | 4.20 | 10.23 | 38 |
| 7.6 | Co(OOC(CH₂)₁₆CH₃)₂ | 5.00 | 10.35 | 6 |

The results of the tests indicate that stearates of cations characterized by a logβ_{DBM} comprised between 8.67 (Cd⁺⁺) and 10.23 (Zn⁺⁺) have a significant activity also at 130° C.

### Example 7

### Test for Esterification of Oil with Low Content of Acidity with Pb Stearate in an Autoclave at 150-160° C.

A reaction test was conducted by loading into an agitated autoclave with a capacity of 1 liter 250 g of soybean oil with an acidity of 0.2% by weight, 110 g of methanol, and 5.61 g of catalyst (Pb stearate).

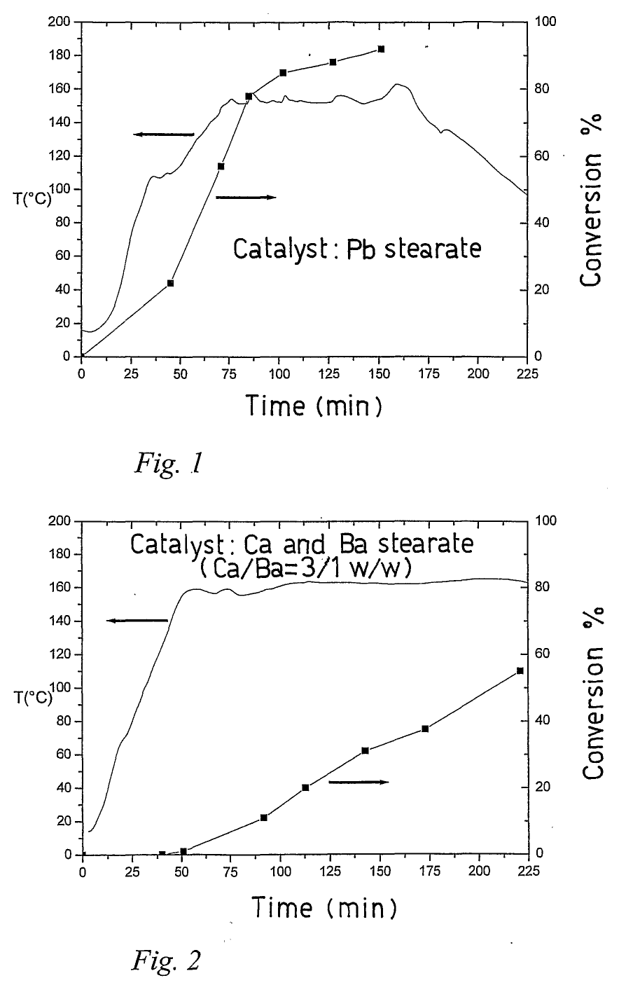

The autoclave was heated in 60 minutes up to 150-160° C. and kept at this temperature for 100 minutes and then cooled to ambient temperature. The temperature profile used is given in FIG. 1.

During the test, samples were taken at various times and analyzed using the H-NMR technique [10]. The results are given in FIG. 1. As can be seen, after 160 minutes of reaction, 92% conversion of the oil to methyl ester was achieved.

### Example 8

### Test for Esterification of Oil with Low Content of Acidity with Ca and Ba Stearate in an Autoclave at 150-160° C.

A reaction test was conducted by loading into an agitated autoclave with a capacity of 1 liter 247 g of soybean oil with an acidity of 0.2% by weight, 111 g of methanol, and 5.65 g of catalyst (catalyst claimed by Basu and Norris [7]; calcium acetate and barium acetate, Ca/Ba=3/1 w/w).

The autoclave was heated in 50 minutes up to 150-160° C. and kept at this temperature for 170 minutes and then cooled to ambient temperature. The temperature profile used is given in FIG. 2.

During the test, samples were taken at various times and analyzed using the H-NMR technique [10]. The results are given in FIG. 2. As can be seen, after 220 minutes of reaction, 55% conversion of the oil to methyl ester was achieved.

By comparing the results of Examples 7 and 8, it is confirmed immediately and unequivocally that Pb stearate is distinctly more active than the catalyst claimed by Basu and Norris [7].

### Example 9

### Test for Esterification of Oil with High Content of Acidity with Pb Stearate in an Autoclave at 220° C.

A reaction test was conducted by loading into an agitated autoclave with a capacity of 1 liter 231.5 g of soybean oil, 18.5 g of oleic acid (oil with initial acidity equal to 7.4% w/w), 114 g of methanol, and 0.1 g of catalyst (Pb stearate).

The autoclave was heated in 80 minutes up to 220° C. and kept at this temperature for 200 minutes and then cooled to ambient temperature. The product discharged from the autoclave was filtered. The methanol was distilled and the glycerol phase was separated from the ester phase by means of a separator funnel. The ester fraction was placed in contact for 1 hour with 6 g of Amberlyst-15 resin in a round-bottomed flask under slight agitation at ambient temperature in order to eliminate the lead. The resin was removed by filtration. The result is an ester phase with a residual acidity, determined by titration, equal to 0.74% by weight [11], a global conversion to methyl esters determined by NMR equal to 96%, and a concentration of Pb⁺⁺ of 4 ppm determined by atomic absorption.

### Example 10

### Test for Esterification of Oil with High Content of Acidity with Pb Stearate in an Autoclave at 150-160° C.

A reaction test was conducted by loading into an agitated autoclave with a capacity of 1 liter 237.5 g of soybean oil, 12.5 g of oleic acid (oil with initial acidity equal to 5% w/w), 114 g of methanol, and 1 g of catalyst (Pb acetate).

The autoclave was heated in 60 minutes up to 150-160° C. and kept at this temperature for 350 minutes and then cooled to ambient temperature. The product discharged from the autoclave was filtered. The methanol was distilled and the glycerol phase was separated from the ester phase by means of a separator funnel. The ester fraction was placed in contact for 1 hour with 8 g of Amberlyst-15 resin in a round-bottomed flask under slight agitation at ambient temperature in order to eliminate the lead. The resin was removed by filtration. The result is an ester phase with a residual acidity, determined by titration, equal to 1% by weight [11], a global conversion to methyl esters determined by NMR equal to 96%, and a concentration of Pb⁺⁺ of 3 ppm determined by atomic absorption.

In the case of Pb stearate, it is possible to work at temperatures lower than, or equal to, 160° C. and therefore at a pressure lower than 20 bars, whereas for the catalysts proposed by Basu, in order to have an activity that is useful from the point of view of the process, it is necessary to work at temperatures above 200° C. and therefore at pressures exceeding 40 bars.

### REFERENCES

[1] U.S. Pat. No. 4,164,506 (1979)
[2] U.S. Pat. No. 5,730,029 (1998)
[3] U.S. Pat. No. 4,695,411 (1987)
[4] U.S. Pat. No. 4,698,186 (1987)
[5] Zhang, Y., Dubé, M. A., McLean, D. D., Kates, M. Bioresource Technology, 2003, 89, 1-16
[6] U.S. Pat. No. 5,713,965
[7] U.S. Pat. No. 5,525,126 (1994)
[8] Van Uitert, L. G., Femelius, C., Douglas, B. E., J. of Am. Chem. Soc. 1953, 75, 2736
[9] Tomita, K., Ida, H. Polymer, 1975, 16, 185
[10] Gelbard, G., Brès, O., Vargas, R. M., Vielfaure, F., Schuchardt, U. F. JAOCS, 1995, 72, 1239
[11] ASTM D803-82 (colourimetric method)

The disclosures in Italian Patent Application no. M12004A001323, from which this application claims priority, are incorporated herein by reference.

What is claimed is:

1-11. (canceled)

12. A process for producing esters from vegetal oils and animal fats, comprising the step of transesterification of vegetal oils or animal fats by reaction with an alcohol with low molecular weight in the presence of a catalyst comprising a salt of a carboxylic acid with a metal, characterized in that said salt of a carboxylic acid with a metal is a salt of a carboxylic acid with a metal selected from the group consisting of metals having a stability constant of the complex with di-benzoyl-methane logβDBM in the range between 8.54 and 10.35, or is a salt with a metal of a carboxylic acid selected from the group consisting of fatty acids.

13. The process according to claim 12, wherein said salt of a carboxylic acid with a metal is a salt of a carboxylic acid with the metal selected from the group consisting of metals having a stability constant of the complex with di-benzoyl-methane logβ_{DBM} in the range between 8.54 and 10.35.

14. The process according to claim 12, wherein said salt of a carboxylic acid with a metal is a salt with a metal of a carboxylic acid selected from the group consisting of fatty acids.

15. The process according to claim 12, wherein said metal is selected from the group consisting of Mg, Cd, Mn, Pb, Zn, Co.

16. The process according to claim 15, wherein said metal is selected from the group consisting of Cd, Mn, Pb and Zn.

17. The process according to claim 16, wherein said metal is Pb.

18. The process according to claim 12, wherein said carboxylic acid is a fatty acid.

19. The process according to claim 18, wherein said fatty acid is stearic acid.

20. The process according to claim 19, wherein said salt is lead stearate.

21. The process according to claim 12, wherein said alcohol is selected from the group consisting of ethanol and methanol.

22. The process according to claim 12, wherein said reaction occurs at a temperature comprised between 100 and 260° C.
